(19) United States
(12) Patent Application Publication
(10) Pub. No.: US 2022/0401749 A1
(43) Pub. Date: Dec. 22, 2022

(54) DEVICE PROVIDING NEUROTHERAPEUTIC INTERVENTION VIA MULTI-MODAL ENERGY THERAPY

(71) Applicant: Rowan University, (US)

(72) Inventor: Charles D. MCGLYNN, Williamstown, NJ (US)

(21) Appl. No.: 17/830,888

(22) Filed: Jun. 2, 2022

Related U.S. Application Data
(60) Provisional application No. 63/213,360, filed on Jun. 22, 2021.

(51) Int. Cl. A61N 2/00; A61N 5/06; A61H 23/02

(52) U.S. Cl. CPC A61N 2/002; A61N 2/006; A61N 5/0622; A61H 23/0236; A61N 2005/0659; A61N 2005/0663; A61N 2005/0626; A61H 2201/1607; A61H 2201/165; A61H 2201/10; A61N 2005/0647; A61H 2205/02

(57) ABSTRACT

The present invention relates generally to a device for providing multi-modal energy therapy to treat neurogenerative disorders, and more particularly to a device providing multi-modal neuro-supportive energy therapy to a person via a daily session in a single facility. The device provides safe, well studied energy therapies in a controlled manner to stimulate neuronal mitochondrial activity which powers the brains neurons and is often defective in neurodegenerative disease states. Selected energy delivery mechanisms and frequencies are employed to stabilize microglial activity and promote microglial homeostasis and neuroprotective secretions, including mitochondrial melatonin and other neuroprotective factors. In addition to practicality, the combination of these proven therapeutic modalities into a single device may offer synergies not found when they are employed alone. This focused combination of these unique energy based therapies may offer new hope to slow, stop, reverse or mitigate neurogenerative disorders, such as Alzheimer's, ALS and Parkinson's diseases.

### CROSS-REFERENCE TO RELATED APPLICATION

This application claims the benefit of priority, under 35 U.S.C. § 119(e), of U.S. Provisional Patent Application No. 63/213,360, filed Jun. 22, 2021, the entire disclosure of which is hereby incorporated herein by reference.

### FIELD OF THE INVENTION

The present invention relates generally to a device for providing multi-modal energy therapy to treat neurogenerative disorders, and more particularly to a device providing multi-modal neuro-supportive energy therapy to a person via a daily session in a single facility.

The apparatus provides an array of safe, well studied energy therapies in a controlled, sequential manner to stimulate neuronal mitochondrial activity which powers the brains neurons and is often defective in neurodegenerative disease states. Selected energy delivery mechanisms and frequencies are also employed to stabilize microglial activity and promote microglial homeostasis and neuroprotective secretions.

In addition to practicality, the combination of these proven therapeutic modalities into a single device may offer synergies not found when they are employed alone. This focused combination of these unique energy based therapies may offer new hope to slow, stop, reverse or mitigate neurogenerative disorders, such as Alzheimer's, ALS and Parkinson's diseases.

### BACKGROUND

Energy therapy has been around for millennia. Acupuncture is the most well-known and perhaps the oldest energy therapy and its usefulness continues to be demonstrated around the world. The brain's neurons at rest have a voltage of about 70 millivolts and neurons can be considered batteries due to their ability to generate a charge across its membrane. This difference in electrical potential when divided by the microscopic distance traversed results in about 14 million volts per meter or 4× the force needed to produce lightning. Thus, the potential for energy therapy for the brain is a very inviting target of study.

Much has been discovered about our genetic code and all manner of cellular processes, but what Albert Szent-Gyorgyi noted decades ago, very little is known about “basic life functions including sleep, pain, the control of cell differentiation, growth and healing”. Another Hungarian physician, Dr. Oskar Estebany, noted that horses that he groomed recovered better than other horses due to the healing power of touch. This was discovered before the human magnetic field could even be measured.

Inflammation has long been recognized as a precursor to many bodily ailments and has now been identified as a potential prime mover in neurodegenerative disease as well. Our device is based the ideas of scientists who have demonstrated that minute—low frequency signals can impart an outsized effect on cellular activities through altering signaling pathways while providing external stimuli to the cell's interior. Weak signals may be amplified by cellular membranes which have protruding proteins that Dr. Ross Adey described as “a field of waving corn, responding to an infinite variety of faint electrochemical breezes that blow along the membrane surface” The brain's pineal gland has been demonstrated to be sensitive to earth's geomagnetic field and even the earth's Schumann resonances—which are often referred to as earth's pulse—affect the brain's melatonin levels.

Such hormonal responses to electromagnetic fields are a proposed mechanism of action for our device. Theories of cooperativity that explain how a system can transition from one stable state to another via different mechanisms and at different speeds such as phase transitions, hysteresis and avalanche effects can be induced from weak signals through an amplification effect that may take place suddenly or gradually. We believe that such an amplification effect may well be induced via the specific combination of weak signals we envision.

The device is focused on three complimentary areas of supporting the brain's roughly 86 billion neurons. First by stimulating their mitochondrial energy. Mitochondria are cellular organelles that provide energy for cellular function. Mitochondria are present in all cells except red bloods cells, usually a couple of hundred thousand per cell, but this number falls with age. They use oxygen to turn the food we eat into energy during normal cellular respiration, but during this process damaging free radicals area also formed as a byproduct. In the brain mitochondrial presence is an order of magnitude higher at an estimated 2 million per cell. In certain neurodegenerative diseases these numbers can suddenly begin to fall dramatically with significant deleterious effects on cellular function that manifest as disease as critical minimum thresholds are breached.

The brain's mitochondria supply its 86 billion neurons with energy via oxidative phosphorylation of glucose or ketone bodies if glucose is lacking. If this metabolism begins to fail universally or in selected regions of the brain, symptoms of neurodegenerative disease will begin to appear. Various energy therapies are known in the healing arts. Each energy therapy, taken alone, is understood or believed to provide various different beneficial effects to the human body, including pain relief, mental relaxation, improved mood, nitric oxide production and improved circulation, tissue repair and improved hormonal secretions including pituitary and adrenal support. For example, red light photons are absorbed by the mitochondria and stimulate ATP production within them. Near red light energy has also been demonstrated to improve cellular communication and reduce oxidative stress at the cellular level. Another pathway by which energy therapies support cellular function is cytochrome c oxidase. Cytochrome c oxidase is a mitochondrial enzyme that is excited when near infrared photons energy fields reach it. When cytochrome c oxidase is produced, ATP is production is enhanced and signaling molecules are sent out that direct cellular repair and free radical scavenging. These mitochondrial processes can also be stimulated by other subtle forms of energy including magnetic pulses, electrical microcurrents and certain sound frequencies. But while anecdotal evidence exists for many energy therapies—including acupuncture, little is known about their potential for beneficial therapeutic effects on neurogenerative disorders such as Alzheimer's, amyotrophic lateral sclerosis (ALS) and Parkinson's diseases. The second function is through supporting homeostasis within the brain's immune system which is regulated by the microglia. The microglia act as the brain's macrophages and can destroy foreign invaders. But when the microglia become inflamed due to genetic predisposition, environmental factors such as stress or toxins their activity can upregulated so high that they begin to destroy healthy brain tissue including the brain's synapses. Just as like an autoimmune disorder such as rheumatoid arthritis symptoms occur when healthy tissue such as cartilage is destroyed by macrophages.

In microglial inflammation the disturbed microglia inflame the brain with cytokines and other neuroinflammatory chemicals which actively destroy the brain's synapses, yet under normal conditions they protect these critical junctions from various potential disturbances by releasing protective chemicals and signals for helpful proteins. They can also secrete nutrients to stimulate the creation of healthy new neurons and create new synapses and help sick neurons heal and grow new neuronal projections. A key to solving neurodegenerative disease must involve controlling the microglia when they are in an inflamed state and harnessing their healing powers once they are stabilized.

The third function is to increase the production of melatonin within the neuronal mitochondria. Mitochondrial melatonin acts to prevent premature cellular death. The only known way to raise intracellular mitochondrial melatonin levels is to stimulate the mitochondria to produce it themselves. Recently scientists have estimated that up to 95% of the human body's melatonin is produced inside its mitochondria where it functions to cool them down by scavenging free radicals. This allows the mitochondria to work efficiently and increases their vitality. This intracellular melatonin cannot be measured in blood and cannot be supported by oral melatonin supplements.

The fourth function is to promote communication through the brain's white matter. If subtle energy fields can augment communication along the brain's 85,000 mile long axonal highway, theoretically brain function should be improved. The major component of the brain's white matter are bundles of axons surrounded by myelin sheathing that speeds up this communication system by an estimated 100×. These axons connect neurons to each other via synapses. The extracellular space (ECS) plays a role here too. In Alzheimer's and in migraines it contracts, which hinders signaling—one permanently, the other temporarily. It has only been very recently that such white matter features have been able to be imaged in real time. In the future studies will need to be done to reveal the effect of subtle energy fields on the ECS and white matter.

What is needed is a device for providing energy therapy to slow, stop, reverse or mitigate neurogenerative disorders. Such a device could be complementary to nutritional and medicinal therapies.

### SUMMARY OF THE INVENTION

The present invention provides a device providing targeted, multi-modal neuro-supportive energy therapy for neuronal mitochondria and support for microglial homeostasis to a person, to slow, stop, reverse or mitigate neurogenerative disorders, such as Alzheimer's, ALS and Parkinson's diseases. Accordingly, the present invention relates generally to a device for providing multi-modal energy therapy to treat neurogenerative disorders, and more particularly to a device providing neurotherapeutic intervention via multi-modal energy therapy stimulating neuronal mitochondrial respiration, mitochondrial melatonin product, and the promotion of microglial supported synaptic plasticity and the release of neurotrophic factors, and suitable for providing multi-modal neuro-supportive energy therapy to a person via a daily session in a single facility.

The brain has been demonstrated to be highly plastic and relatively more responsive to stimuli under certain conditions. In part, the device of the present invention is operative to stimulate mitochondrial to produce intracellular mitochondrial melatonin. Further, the device is designed to cause the patient to enter a theta wave state prior to the delivery of the neuro-supportive therapies so that the neuronal response will be optimized. Since people may respond to these therapies in a highly individualistic manner, the device of the present invention offers a broad suite of complimentary energy therapies to help meet each individual's needs and ensure a higher success rate than might be possible with any of these therapies alone.

The device provides an array of safe, well-studied energy therapies in a controlled, sequential (or concurrent) manner to stimulate neuronal mitochondrial activity which powers the brains neurons and is often defective in neurodegenerative disease states. Selected energy delivery mechanisms and frequencies are also employed to stabilize microglial activity and promote microglial homeostasis and neuroprotective secretions.

In addition to practicality, the combination of these proven therapeutic modalities into a single device may offer synergies not found when they are employed alone. This focused combination of these unique energy based therapies may offer new hope to slow, stop, reverse or mitigate neurogenerative disorders, such as Alzheimer's, ALS and Parkinson's diseases.

### BRIEF DESCRIPTION OF THE FIGURES

The following detailed description of various embodiments of the invention will be better understood when read in conjunction with the appended drawings. For the purpose of illustrating the invention, certain embodiments are shown in the drawings. It should be understood, however, that the invention is not limited to the precise arrangements and instrumentalities of the embodiments shown in the drawings.

FIG. 1 is front view of a helmet device providing multi-modal neuro-supportive energy therapy in accordance with an exemplary embodiment of the present invention;

FIG. 2 is front view of a plate device providing multi-modal neuro-supportive energy therapy in accordance with another exemplary embodiment of the present invention; and

FIG. 3 is front view of a chair device providing multi-modal neuro-supportive energy therapy in accordance with yet another exemplary embodiment of the present invention;

FIG. 4 is a front view of a two-part wearable accessory system providing multi-modal neuro-supportive energy therapy in accordance with yet another exemplary embodiment of the present invention; and

FIG. 5 is a perspective view of the helmet of FIG. 4.

### DETAILED DESCRIPTION

Devices in accordance with the present invention include multiple different types of stimulators for delivering multiple different types of energy therapies to the body, thereby collectively providing multi-modal neuro-supportive energy therapy. The devices thereby combine several existing therapeutic modalities into a single device to provide a benefit to users/patients through the synergy of compounded energy-based therapies.

The device provides structure for delivering these multi-modal neuronal mitochondria supportive energy therapies and microglial homeostatic promotion directly to the head, which is believed to be important and relatively more effective for neurogenesis and mitogenesis by promoting an energized yet restful, steady state in the brain. The restful phase may be promoted via 40 hz light therapy and binaural beats audio therapy while energy therapy is delivered in the form of red light, magnetic pulses, Solfeggio frequency tones and microcurrents.

The device benefits users/patients through ease of use, and time savings by, at least in some embodiments, delivering multiple different energy therapies to a user/patent concurrently. By intervening from several different energy therapy pathways, it is believed possible to help a wider group of patients than any single energy-based therapy could. Further, the multiple energy therapy pathways offered are believed to be synergistic, such that the result of the combined therapies outweighs the benefits of each individual therapy provided separately.

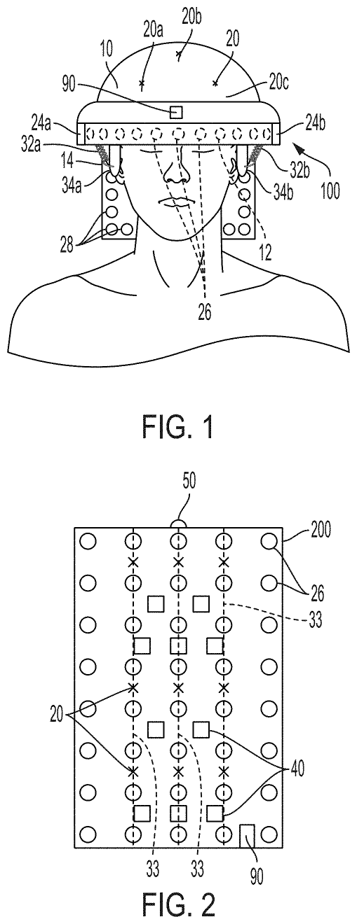

Referring now to FIG. 1, an exemplary helmet device 100 in accordance with the present invention is shown. In accordance with the present invention, the helmet 100 includes multiple different types of stimulators for delivering multiple different types of energy therapies to the body, thereby collectively providing multi-modal neuro-supportive energy therapy. More particularly, the helmet 100 includes a cap portion 10 sized and shaped to fit over and be worn upon a range of sizes of human heads.

At least one magnetic pulser stimulator 20 is supported on the cap portion 10 of the device, overlying an upper portion of the skull. Magnetic pulser stimulator devices are known in the art for providing pulsed electromagnetic field (PEMF) therapy, which is generally regarded as a drug-free, non-invasive, pain-relief alternative. Such magnetic pulsers create/deliver microcurrents of direct current electricity (low level electromagnetic radiation) that work with the body's natural magnetic and/or electric fields. These magnetic pulsers generally operating the same low frequency range as the earth's magnetic field, to help restore a person's own magnetic field to its natural state. It is believed that these magnetic pulsers may be used to deliver energy therapy to mitochondria, to stimulate tissue growth, and particularly to stimulate and/or prompt regeneration of nerve tissue to counteract nerve tissue degradation associated with neurogenerative diseases.

The frequency (e.g., 5-30 Hz), intensity and/or other parameters may be varied to provide a desired effect, e.g., to modify or control a depth of penetration of the energy into bodily tissue. In certain embodiments, multiple magnetic pulser stimulators 20a, 20b, 20c may be arranged on the helmet in spaced positions and orientations about the cap portion 10, and the frequency, intensity and/or other parameters may be varied to cause radiated energy at a less-than-therapeutic level to intersect and/or combined at a desired physical location (e.g., depth) with the head of the wearer, such that combined energy from the multiple stimulators provides a therapeutic level of energy, to allow for targeted delivery of energy to internal regions of the skull. The device can restore membrane potential which facilitates cellular hydration, reduces inflammation and improves circulation. The magnetic pulser(s) 20, 20a, 20b, 20c are coupled to a controller 90 supported on the helmet. The controller 90 comprises a device that sends out magnetic waves in a pulsed form. The frequency and strength of the pulses can both be regulated to produce the desired therapeutic effect.

The helmet 100 further includes a visor portion 12 extending downwardly from the cap portion 10. The visor portion 12 is sized and shaped to cover the forehead and eyes or the wearer when the cap portion 10 is worn on the head. The visor 12 provides physical structure to block/obscure at least a portion of the field of view of the wearer, and further provides supportive structure for mounting of stimulators. In the embodiment shown in FIG. 1, the visor 12 supports a pair of light sources 24a, 24b. The light sources 24a, 24b are coupled to the controller 90 and the controller 90 is configured to provide light as desired to provide a desired therapeutic effect. Preferably, the controller is configured to cause the light sources 24a, 24b to provide flickering light at a frequency in a frequency range of about 20 Hz to about 50 Hz, and preferably about 40 Hz, which has been shown to impact gamma brain waves, which are associated with Alzheimer's disease.

Notably, it is believed that disturbances to gamma brain waves result in an increased buildup of plaque protein between brain cells, which is an indication of Alzheimer's disease. Gamma brain waves are known to typically oscillate between 20 Hz and 50 Hz, and exposure to flickering light in the range of about 20 Hz to about 50 Hz, and preferably about 40 Hz, is believed to improve gamma brain waves and reduce plaque buildup by increasing the production of microglia, which is believed to be the main immune cell of the brain.

In a preferred embodiment, the light sources 24a, 24b are positioned on the visor so as to be located in a region of peripheral vision of the wearer. For example, separation of the light sources by an angle of about 90 to about 70 degrees relative to a center point of the helmet has been found suitable for this purpose. Positioning of the 40 Hz light sources in the peripheral vision region is beneficial in that by avoiding a direct field of view this frequency in induces a state of deep relaxation and has been demonstrated to improve memory and relaxation in a variety of individuals and is being studied in Alzheimer's currently.

The visor 12 further provides physical structure for mounting of stimulators. In the embodiment shown in FIG. 1, the visor 12 supports red and near infrared (NIR) light sources 26. The light sources 26 are coupled to the controller 90 and the controller 90 is configured to provide light as desired to provide a desired therapeutic effect. Preferably, these light sources are configured to provide light at a frequency in a wavelength range of about 850 nm and 660 nm. It has been shown exposure to light energy in the red and near infrared ranges are effective in providing red light therapy effects that improve cellular function and improve health and wellness. Preferably, these light sources 26 are positioned on the visor 12 (and/or cap 10) to overlie, and thus emit light upon, an exposed forehead region (outside of the hairline) of the wearer, so as to avoid the blocking of emitted light by the wearer's hair.

The helmet 100 further includes a neck portion 14 extending downwardly from the cap portion 10. The neck portion 14 is sized and shaped to overlie and span the neck of the wearer, preferably below a region of a likely hairline. The neck portion 14 thereby provides physical structure supporting additional red and near infrared (NIR) light sources 28. This allows for the provision of direct red light therapy to the cerebellum at a greater dose than can be achieved if the frontal lobe must be penetrated. Similarly, the light sources 26 are coupled to the controller 90 and the controller 90 is configured to provide light as desired to provide a desired therapeutic effect.

The helmet 100 further includes a pair of spaced loudspeaker 30a, 30b positioned to be near each ear of the wearer, e.g. supported on the cap portion 10. As known in the art, audio therapy in the nature of binaural beats is effectively an illusion created by the brain when a person listens to two tones with slightly different frequencies in different ears at the same time. The difference in frequencies creates an illusion of a third sound, which is a rhythmic beat. The loudspeakers 30a, 30b are coupled to the controller 90 and the controller 90 is configured to provide binaural beats audible signals via the loudspeakers 30a, 30b. Binaural beats are recognized to synchronize hemispheric or other brain activity, and are understood to cause neurons throughout the brain to begin sending electrical messages at the same rate as the imagined rhythmic beat. Exposure to binaural beats is understood to decrease anxiety, increase focus, and improve mood, and in any event, provide neural stimulation to the brain that is believed to be beneficial to those suffering from neurodegenerative diseases. The neck portion may also include at least one magnetic pulser.

In certain embodiments, the binaural beats audio therapy involves delivering periodic tones at the Solfeggio frequencies, and more particularly at about 285 Hz and 528 Hz. The Solfeggio frequencies are set of frequencies known for promoting well-being and healing in the body. The 285 Hz and 528 Hz bands are widely used for tissue healing and cellular rejuvenation. In certain embodiments, binaural beats or other tones at the Solfeggio frequencies are provided prior to red light, magnetic pulse and/or microcurrent energy therapies. In certain embodiments, binaural beats or other tones at the Solfeggio frequencies are provided after red light, magnetic pulse and/or microcurrent energy therapies.

The helmet 100 further includes a pair of electrical leads 32a, 32b terminating in conductive ear clips 34a 34b. The leads and ear clips are coupled to the controller 90 and the controller 90 is configured to deliver low voltage microcurrent at about one millionth of an ampere. This level of energy will likely not be felt by detected by the recipient and has been demonstrated to be quite safe. By comparison, a TENS unit employs up to 100 milliamps. The energy in this unit is directed to the earlobes (to which the ear clips are clipped) as part of microcurrent therapy. Microcurrent therapy generally involves the delivery of low-voltage current similar to the natural electrical currents in the body. The microcurrents products are understood to increase circulation, promote collagen and elastic product in the skin, dramatically increase adenosine triphosphate (ATP), improve fibroblast activity for collagen synthesis, and increase amino acid production. Notably, such microcurrents are believed to be operative at 10 Hz and 40 Hz may be applied with moistened padded ear clips thus to be beneficial to those suffering from neurodegenerative diseases. 40 Hz has been shown to reduce neuronal inflammation by suppressing microglial inflammatory cytokines.

Each of the stimulators discussed above are operative to stimulate the human body, and more particularly to deliver energy to the head (including neck) regions in tandem. Delivery directly to the head and neck is beneficial because we wish to target the neuronal mitochondria and microglia directly and sequentially. Further, delivery in tandem (either in sequence and/or simultaneously) is believed to provide for a synergistic effect in which the receipt of the different therapies in tandem provides a greater therapeutic benefit to the patient than any therapy alone, or in multiple therapies that are delivered separately (in time) but not sufficiently close in time to be in tandem such that they combine to provide a therapeutic effect in the same manner as many multi-drug and nutritional therapies are directed.

Referring now to FIG. 2, an exemplary torso plate device 200 in accordance with the present invention is shown. In accordance with the present invention, the torso plate 200 includes multiple different types of stimulators for delivering multiple different types of energy therapies to the body, thereby collectively providing multi-modal neuro-supportive energy therapy. More particularly, the exemplary torso plate 200 includes a plurality of magnetic pulsers 20 and a plurality of red and near infrared light sources 26. The magnetic pulsers 20 and red/near infrared light sources 26 are coupled to the controller 90 and the controller 90 is configured to provide magnetic pulses and light emission to provide a desired therapeutic effect. The magnetic pulsers 20 and/or the red/near infrared light sources 26 may be arranged in one or more regularly-spaced arrays over the surface area of the torso plate 200. The rationale for this embodiment is to target to the brain-gut signaling pathways which are now believed to be involved neurodegenerative disorders. In a preferred embodiment, the magnetic pulsers 20 and light sources 26 are provided on the torso plate device in central locations, primarily or exclusively, selected to abut and/or align with the spinal cord of a person's torso that is in an expected alignment with the torso plate device.

Additionally, the exemplary torso plate 200 includes a plurality of speakers 40 providing a biofield tuning therapeutic effect. Biofield tuning involves delivery of sound waves/vibrations, not necessarily audible, that are consistent with the body's natural magnetic field. The application of Solfeggio frequencies has been demonstrated to have a positive effect on a variety of human tissues including bones and muscles often via tuning forks which the system mimics. Clinical studies have evaluating biofields show benefits including alleviating anxiety and increasing heart rate variability (HRV) this supports homeostasis within the microglia and will save synapses from destruction among other benefits. The delivery of an audio signal consistent with the body's natural magnetic field is believed to promote restoring of the body's biofield to its natural health state, perhaps by way of resonance. The speakers 40 are coupled to a controller and the controller is configured to provide sound waves operable to provide a desired therapeutic effect. The controller may be similar to the controller 90 described above. The controller may be separate from the controller 90 described above, or both the helmet 100 and the torso plate 200 may share a single controller 90 and be configured to operate in tandem. The speakers 40 may be arranged in a regularly-spaced array over the surface area of the torso plate 200. In certain embodiments, one or more speakers 40 may positioned to aligned with the spinal cord during use of the torso plate device 200, and the controller 90 may be configured to cause those speakers 40 to deliver tones at the Solfeggio frequencies, to stimulate the spinal cord with energy/vibrations at the Solfeggio frequencies.

Additionally, the torso plate 200 may include microcurrent wires 33 that lie close to the surface of the torso plate 200 for being in close proximity to the skin to create/deliver microcurrents of direct current electricity (low level electromagnetic radiation) that work with the body's natural magnetic and/or electric fields. Three such wires are shown in FIG. 2, in dashed line.

The torso plate may include a hook or loop 50 adapted for mounting of the torso plate 200 to a chair, etc.

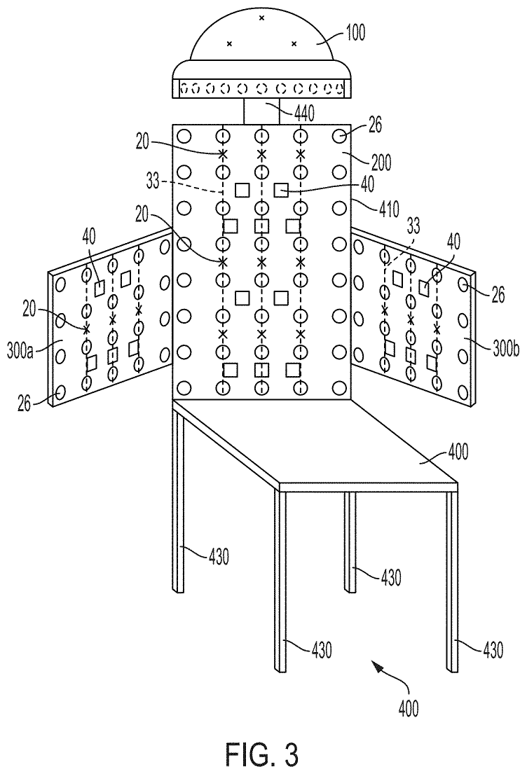

Referring now to FIG. 3, an exemplary chair device 400 in accordance with the present invention is shown. In accordance with the present invention, the chair 400 includes multiple different types of stimulators for delivering multiple different types of energy therapies to the body, thereby collectively providing multi-modal neuro-supportive energy therapy. More particularly, the chair 400 includes a backrest 410 adjacent a seat 420 supported on legs 430. A torso plate 200 similar to that shown in FIG. 2 is supported on the backrest 410 of the chair. A helmet 100 similar to that shown in FIG. 1 is supported above the backrest 410 on a mount 440, which may allow for pivotable movement of the helmet 100 between a raised/inoperative position and a lowered/operative position, somewhat like a conventional hairdresser salon chair with a head/hair dryer attachment.

The exemplary chair 400 of FIG. 3 further includes a pair of side flaps 300a, 300b, positioned to be adjacent the arms of an occupant seated in the chair 400. The side flaps include one or more of any of the stimulators described above, which are connected to the controller 90 for delivering energy therapies in tandem, to the arms, as desired. This type of arrangement is believed to be advantageous in that it provides physical structure allowing energy therapies to be delivered in tandem over a relatively greater portion of the user's body, namely, to the head, torso and arms in tandem, such as concurrently in a single session (which may be, for example, a 60 minute session in which each of the helmet, torso plate and side flaps are operative for 20 minutes, successively), or simultaneously (e.g., in a single 20 minute session in which the helmet, torso plate and side flaps are operative simultaneously).

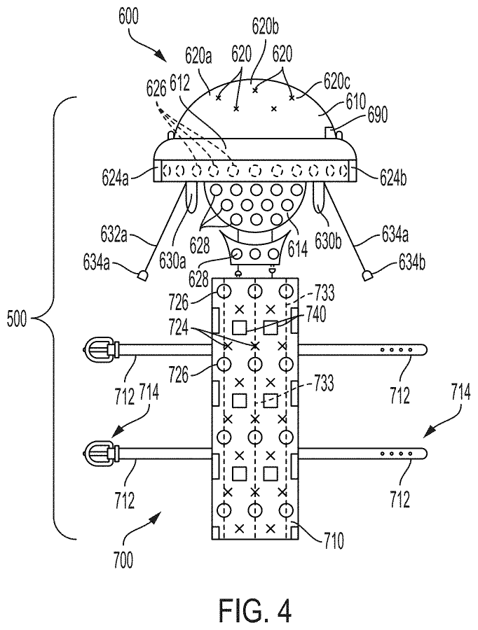

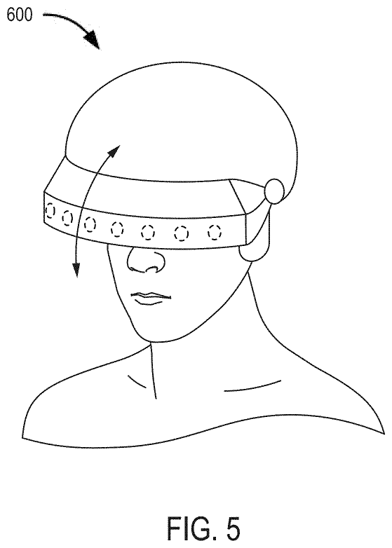

FIG. 4 is a front view of a two-part wearable accessory system 500 providing multi-modal neuro-supportive energy therapy in accordance with yet another exemplary embodiment of the present invention. As shown in FIG. 4, the wearable accessory system 500 includes a helmet 600 and a spinal appliance 700. FIG. 5 is a perspective view of the helmet 600 of FIG. 4.

The helmet 600 of FIG. 4 is somewhat similar to the helmet 100 of FIG. 1. Accordingly, the helmet 600 includes multiple different types of stimulators for delivering multiple different types of energy therapies to the body, thereby collectively providing multi-modal neuro-supportive energy therapy. More particularly, the helmet 600 includes a cap portion 610 sized and shaped to fit over and be worn upon a range of sizes of human heads.

At least one magnetic pulser stimulator 620 is supported on the cap portion 610 of the device, overlying an upper portion of the skull. The frequency (e.g., 5-30 Hz), intensity and/or other parameters may be varied to provide a desired effect, e.g., to modify or control a depth of penetration of the energy into bodily tissue. In certain embodiments, multiple magnetic pulser stimulators 620a, 620b, 620c may be arranged on the helmet in spaced positions and orientations about the cap portion 610, and the frequency, intensity and/or other parameters may be varied, as described above. The magnetic pulser(s) 620 are coupled to a controller 690 supported on the helmet. The controller 690 comprises a device the sends out magnetic waves in a pulsed form. The frequency and strength of the pulses can both be regulated to produce the desired therapeutic effect.

The helmet 600 further includes a visor portion 612 extending downwardly from the cap portion 10. Unlike the helmet described above, in this embodiment of the helmet 600, the visor portion 612 is movably mounted (e.g., via pivot pins) to remainder of the helmet 600 such that the visor portion is movable between a first position and a second position, as will be best appreciated from FIG. 5. The visor portion 612 is sized and shaped to cover the forehead and eyes of the wearer when the cap portion 610 is worn on the head when the visor is in the first (operable) position. The visor 612 thereby provides physical structure to block/obscure at least a portion of the field of view of the wearer, and further provides supportive structure for mounting of stimulators. In the embodiment shown in FIG. 4, the visor 612 supports a pair of light sources 624a, 624b. Preferably, the light sources are positioned to be located adjacent the peripheral edges of the field of vision of a wearer of the helmet 600. For example, separation of the light sources by an angle of about 90 to about 70 degrees relative to a center point of the helmet has been found suitable for this purpose. The light sources 624a, 624b are coupled to the controller 690 and the controller 690 is configured to provide light as desired to provide a desired therapeutic effect, as described above. The visor portion is further moveable to the second (inoperative) position in which the visor no longer covers the eyes of the wearer when the cap portion 610 is worn on the head, so that the visor may be positioned such that the wearer's vision is not obscured, e.g., while the wearer is not receiving light therapy (e.g., 40 Hz light flashes) via the light sources 624a, 624b on the visor 612.

The visor 612 further provides physical structure for mounting of stimulators. In the embodiment shown in FIG. 4, the visor 612 supports red and near infrared (NIR) light sources 626, e.g., in a wavelength range of about 850 nm and 660 nm. The light sources 626 are coupled to the controller 690 and the controller 690 is configured to provide light as desired to provide a desired therapeutic effect, as described above.

The helmet 600 further includes a neck portion 14 extending downwardly from the cap portion 10, similar to that of the helmet 100 described above. The neck portion 614 is sized and shaped to overlie and span the neck of the wearer, preferably below a region of a likely hairline. The neck portion 614 thereby provides physical structure supporting additional red and near infrared (NIR) light sources 628. Preferably, the neck portion 614 includes light sources 628 positioned to align with/overlie the spinal cord of the wearer of the helmet 600 during use. This allows for the provision of direct red light therapy to the cerebellum and/or spinal cord at a greater dose than can be achieved if the frontal lobe must be penetrated. Similarly, the light sources 628 are coupled to the controller 690 and the controller 690 is configured to control the light sources 628 to emit/provide light as desired to provide a desired therapeutic effect.

In this example, the neck portion 614 includes a suspended rigid portion separate from but flexibly coupled to (e.g., by conductive wires) the remainder of the neck portion. This provides the neck portion 614 with a measure of “flex” or give to allow the suspended portion to move with the body and better confirm to the next in a range of different head/neck positions. The suspended portion may include some of all of the same stimulators (e.g., light sources, microcurrent wires, etc.) as the remainder of the neck portion.

The helmet 600 further includes a pair of spaced loudspeaker 630a, 630b positioned to be near each ear of the wearer, e.g. supported on the cap portion 610. The loudspeakers 630a, 630b are coupled to the controller 690 and the controller 690 is configured to provide binaural beats audible signals via the loudspeakers 630a, 630b. The binaural beats audio therapy may involves delivering periodic tones at the Solfeggio frequencies, and more particularly at about 285 Hz and 528 Hz, as described above. The controller may be configured to provide Binaural beats or other tones at the Solfeggio frequencies prior to and/or after red light, magnetic pulse and/or microcurrent energy therapies, as described above.

Similarly to the helmet 100 described above, this helmet 600 further includes a pair of electrical leads 632a, 632b terminating in conductive ear clips 634a 634b. The leads and ear clips are coupled to the controller 690 and the controller 690 is configured to deliver low voltage microcurrent at about one millionth of an ampere, e.g., from a power source provided on the helmet 600. Notably, such microcurrents are believed to be operative at 10 Hz and 40 Hz may be applied with moistened padded ear clips thus to be beneficial to those suffering from neurodegenerative diseases.

The spinal appliance 700 of FIG. 4 is somewhat similar to the torso plate device 200 of FIG. 2. Accordingly, the spinal appliance 700 includes multiple different types of stimulators for delivering multiple different types of energy therapies to the body, thereby collectively providing multi-modal neuro-supportive energy therapy. More particularly, the exemplary spinal appliance 700 includes a support structure 710. The support structure 710 is generally elongated to span the spinal cord of the wearer, from the neck/shirt collar region to approximately the region of the coccyx. Accordingly, a support structure 710 measuring approximately 16 to 22 inches in length may be suitable. In certain embodiments, support structure 710 of different sizes (e.g., 16, 18, 20 or 22 inches of length) may be provided for use interchangeably with a helmet 600. Similarly, a width of the support structure 710 in the range of approximately 3 to 5 inches in length may be suitable for spanning the spinal cord of the wearer. The support structure may be rigid or non-rigid, but is preferably non-rigid and made of flexible plastic, or a plurality of rigid plastic sections joined by joints or connects to allow the support structure 710 to flex, bend or otherwise confirm to the curvature of the wearer's spine, and/or during use.

The support structure 710 preferably includes at least one belt, strap, tie or the like (collectively, “strap”) that are sufficient in length to collectively encircle a range of common human torso sizes. Further, the strap 712 and/or support structure 710 are provided with complementary fasteners 714, such as hook and loop fastener materials, for securing a strap to the support structure, or for securing complementary straps to each other, to secure the support structure and spinal appliance 700 to the wearer's torso, in a position overlying the wearer's spinal cord.

Similarly to the torso plate 200 described above, the support structure 710 supports a plurality of magnetic pulsers 720 and a plurality of red and near infrared light sources 726, in positions selected to align with wearer's spinal cord when wearing the spinal appliance 700. The magnetic pulsers 720 and/or the red/near infrared light sources 726 may be arranged in one or more regularly-spaced arrays over the surface area of the support structure 710.

Additionally, the support structure 710 may include microcurrent wires 733 that lie close to the surface of the support structure 710 for being in close proximity to the skin to create/deliver microcurrents of direct current electricity (low level electromagnetic radiation) that work with the body's natural magnetic and/or electric fields. Three such wires are shown in FIG. 4, in dashed line.

Additionally, the support structure also supports a plurality of speakers 740 providing a biofield tuning therapeutic effect, as described above. The speakers 40 may be arranged in a regularly-spaced array over the surface area of the support structure 710.

The magnetic pulsers 720, red/near infrared light sources 726, and speakers 740 are coupled to a controller configured to provide magnetic pulses, light emission and an audio biofield tuning signal, to provide a desired therapeutic effect, as described above. In the embodiment shown, they operatively connected to the controller 690 of the helmet 600, by way of wired connections and connectors interconnecting the components of the helmet 600 to the components of the spinal appliance. In an alternative embodiment, the spinal appliance 700 includes a separate controller 700 that may be in communication, e.g., wired or wireless communication, with the controller 690 of the helmet 600. In either case, therapeutic signals can be delivered in a coordinated fashion to the head/neck via the helmet 600, and to the spinal cord via the spinal appliance 700, under the control of the one or more controllers, e.g., 690.

Although the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A multi-model neuro-supportive energy therapy delivery device comprising:
a body sized and shaped to be worn on an anatomical portion of a wearer;
a first stimulator of a first type connected to the controller, the first stimulator being operative to deliver a first energy exposure sequence to provide a first energy therapy to the head; and
a second stimulator of a second type, different from the first type, connected to the controller, the second stimulator being operative to deliver a second energy exposure sequence to provide a second energy therapy to the head, the second energy therapy being different from the first energy therapy;
a controller operatively connected to each of said first stimulator and said second stimulator, said controller being configured to control each of first stimulator and said second stimulator to provide a desired therapeutic effect.

2. The multi-model neuro-supportive energy therapy delivery device of claim 1, wherein at least one of said first stimulator of said first type and said second stimulator of said second type comprises at least one magnetic pulser stimulator.

3. The multi-model neuro-supportive energy therapy delivery device of claim 2, wherein said controller is configured to cause said at least one magnetic pulser stimulator to deliver energy at a selected point within a therapeutic range of frequencies and intensities.

4. The multi-model neuro-supportive energy therapy delivery device of claim 2, wherein at least two of said plurality of magnetic pulser stimulators are arranged in at least one of positions and orientations on the body to cause energy delivered by any one of said plurality of magnetic pulser stimulators at an energy level less than a therapeutic level to combine at a desired physical location to provide a combined energy at a therapeutic energy level at the physical location.

5. The multi-model neuro-supportive energy therapy delivery device of claim 2, further comprising a first plurality of at least one of red and near infrared light sources supported on said body, said at least one of red and near infrared light sources being configured to emit light at a frequency in a wavelength range of about 660 nm to about 850 nm, said first plurality of at least one of red and near infrared light sources being operatively coupled to said controller, said controller being configured to selectively cause said first plurality of at least one of red and near infrared light sources to emit light to provide a desired therapeutic effect.

6. The multi-model neuro-supportive energy therapy delivery device of claim 2, wherein said body comprises a helmet comprising a cap portion sized and shaped to fit on a head of a human, and wherein a plurality of magnetic pulser stimulators is arranged in spaced positions on said cap portion.

7. The multi-model neuro-supportive energy therapy delivery device of claim 6, wherein said helmet further includes a neck portion extending downwardly from said cap portion, said neck portion being sized and shaped to overlie and span a neck of the wearer, said helmet further comprising a second plurality of at least one of red and near infrared light sources configured to emit light at a frequency in a wavelength range of about 660 nm to about 850 nm supported on said neck portion, said at least one of red and near infrared light sources being operatively coupled to said controller, said controller being configured to selectively cause said second plurality of at least one of red and near infrared light sources to emit light to provide a desired therapeutic effect.

8. The multi-model neuro-supportive energy therapy delivery device of claim 6, further comprising a pair of loudspeakers supported on said cap portion, each of said pair of loudspeakers being positioned to be adjacent a respective one of a pair of ears of a wearer of the device, said pair of loudspeakers being operatively coupled to said controller, said controller being configured to selectively cause said pair of loudspeakers to provide an audio signal providing a desired therapeutic effect.

9. The multi-model neuro-supportive energy therapy delivery device of claim 6, wherein said helmet comprises a visor portion extending downwardly from said cap portion, said visor portion being sized and shaped to cover eyes and a forehead of the wearer when the cap portion is worn on a head of the wearer.

10. The multi-model neuro-supportive energy therapy delivery device of claim 9, further comprising a pair of light sources supported on said visor portion at positions selected to be located in a region of peripheral vision of the wearer of the helmet.

11. The multi-model neuro-supportive energy therapy delivery device of claim 9, wherein light sources of said pair of light sources are separated by an angle of about 90 degrees to about 70 degrees relative to a center point of said helmet.

12. The multi-model neuro-supportive energy therapy delivery device of claim 9, wherein said pair or light sources are operatively connected to said controller, and wherein said controller is configured to selectively cause said pair of light sources to emit pulses of visible light to provide a desired therapeutic effect.

13. The multi-model neuro-supportive energy therapy delivery device of claim 9, wherein said controller is configured to cause said pair of light sources to emit pulses of visible light at a frequency in a range of about 20 Hz to about 50 Hz.

14. The multi-model neuro-supportive energy therapy delivery device of claim 9, wherein said visor portion is movably mounted to said cap portion of said helmet, said visor portion being movable between a first position in which the visor portion is positioned to cover the forehead and eyes of the wearer, and a second position in which the visor portion is not positioned to cover the forehead and eyes of the wearer.

15. The multi-model neuro-supportive energy therapy delivery device of claim 9, wherein said body comprises a helmet comprising a cap portion sized and shaped to fit on a head of a human and a visor portion joined to said cap portion, wherein said first plurality of at least one or red and near infrared light sources are supported on said visor portion at positions selected to be located in a region of a forehead of the wearer of the helmet.

16. The multi-model neuro-supportive energy therapy delivery device of claim 1, wherein said body comprises one of a torso plate that is an integral part of a chair adjacent a seat supported on legs, a torso plate that comprises one of a hook, a belt and a loop configured for mounting of the torso plate to a back of a chair, and a spinal appliance comprising a support structure that is elongated to span the spinal cord of the wearer.

17. The multi-model neuro-supportive energy therapy delivery device of claim 16, wherein said support structure comprises at least one of a belt, a strap, and a tie sufficient in length to encircle a range of common human torso sizes, and a fastener for securing said at least one of a belt, a strap and a tie in a position encircling a human torso.

18. The multi-model neuro-supportive energy therapy delivery device of claim 16, wherein each of said first stimulator and said second stimulator is selected from a group consisting of a plurality of magnetic pulsers, a plurality of red or near infrared light sources, a plurality of loudspeakers, and a plurality of electrical leads, each terminating in a clip.

19. The multi-model neuro-supportive energy therapy delivery device of claim 16, wherein said body a pair of side flaps, each of said pair of side flaps comprises at least one of a first stimulator and a second stimulator selected from a group consisting of a plurality of magnetic pulsers, a plurality of red or near infrared light sources, and a plurality of loudspeakers, and a plurality of electrical leads, each terminating in a clip.

20. The multi-model neuro-supportive energy therapy delivery device of claim 1, wherein at least of said first stimulator and said second stimulator comprises the plurality of loudspeakers, and wherein said controller is configured to cause said pair of loudspeakers to provide an audio signal comprising binaural beats.

21. The multi-model neuro-supportive energy therapy delivery device of claim 20, wherein said controller is configured to cause said plurality of loudspeakers to provide an audio signal comprising tones of at least one Solfeggio frequency.

22. The multi-model neuro-supportive energy therapy delivery device of claim 1, further comprising a pair of electrical leads each terminating in a clip, said pair of electrical leads being operatively coupled to said controller, said controller being configured to selectively cause said pair of electrical leads to deliver low voltage microcurrent to provide a desired therapeutic effect.

23. The multi-model neuro-supportive energy therapy delivery device of claim 22, wherein said low voltage microcurrent has an amperage of about one millionth of an ampere, and wherein said low voltage microcurrent is provided at a frequency in a range of about 10 Hz to about 40 Hz.

24. The multi-model neuro-supportive energy therapy delivery device of claim 1, wherein said controller is configured to cause operation of said first stimulator and said second stimulator in one of a sequential fashion and a concurrent fashion.

25. The multi-model neuro-supportive energy therapy delivery device of claim 1, wherein said controller is configured to cause operation of said first stimulator and said second stimulator sequentially.

26. A multi-model neuro-supportive energy therapy delivery device comprising:
a helmet sized and shaped to be worn on a head of a wearer;
a body sized and shaped to be worn adjacent a torso of a wearer;
a first stimulator of a first type connected to the controller, the first stimulator being operative to deliver a first energy exposure sequence to provide a first energy therapy to the head;
a second stimulator of a second type, different from the first type, connected to the controller, the second stimulator being operative to deliver a second energy exposure sequence to provide a second energy therapy to the head, the second energy therapy being different from the first energy therapy; and
a controller operatively connected to each of said first stimulator and said second stimulator, said controller being configured to control each of first stimulator and said second stimulator to provide a desired therapeutic effect.

27. A multi-model neuro-supportive energy therapy delivery device comprising:
a helmet having a cap portion sized and shaped to be worn on a head of a wearer, and a visor portion extending downwardly from said cap portion, said visor portion being movably mounted to said cap portion and being movable between a first position in which the visor portion is positioned to cover a forehead and eyes of the wearer, and a second position in which the visor portion is positioned not to cover the forehead and eyes of the wearer;
a first stimulator of a first type connected to the controller, the first stimulator being operative to deliver a first energy exposure sequence to provide a first energy therapy to the head; and
a second stimulator of a second type, different from the first type, connected to the controller, the second stimulator being operative to deliver a second energy exposure sequence to provide a second energy therapy to the head, the second energy therapy being different from the first energy therapy;
a controller operatively connected to each of said first stimulator and said second stimulator, said controller being configured to control each of first stimulator and said second stimulator to provide a desired therapeutic effect.
